(19) United States
(12) Patent Application Publication
(10) Pub. No.: US 2026/0364466 A1
(43) Pub. Date: Nov. 19, 2026

(54) SYSTEMS AND METHODS FOR FACILITATING DIGITAL INTERACTIONS INVOLVING USER ARBITRATION BETWEEN INDIVIDUAL OUTCOME OBSERVATIONS AND ADVICE

(71) Applicant: CENTRE FOR ADDICTION AND MENTAL HEALTH, TORONTO (CA)

(72) Inventor: ANDREEA OLIVIANA DIACONESCU, VAUGHAN (CA)

(21) Appl. No.: 19/477,195

(22) PCT Filed: Apr. 9, 2024
(86) PCT No.: PCT/CA2024/050450

§ 371 Date: Oct. 21, 2025

Related U.S. Application Data
(60) Provisional application No. 63/461,038, filed on Apr. 21, 2023.

(51) Int. Cl. A63F 13/67; A63F 13/533; G06F 3/04842; G06N 7/01; G06T 13/40; G16H 20/70

(52) U.S. Cl. CPC A63F 13/67; A63F 13/533; G06F 3/04842; G06N 7/01; G06T 13/40; G16H 20/70

(57) ABSTRACT

A game-like environment presented to a user displaying a set of selectable elements. The user is instructed that some selectable elements contain a reward, while others do not. The user is also provided with advice that suggests the selection of one of the selectable elements, and after selection of a particular selectable element, the presence or absence of a reward is revealed. The selectable elements each have a visual attribute selected from a set of visual attributes, enabling the user to develop a sense of which type of visual attribute tends to be rewarding or non-rewarding. The behaviour of the user is modelled using a computational behavioural model, providing model parameters that facilitate tracking of changes in the user's beliefs during gameplay. The probabilistic structure of the game may be altered based on the measured learning performance, as quantified by one or more computational model parameters.

### CROSS-REFERENCE TO RELATED APPLICATION

This application claims priority to U.S. Provisional Patent Application No. 63/461,038, titled “SYSTEMS AND METHODS FOR FACILITATING DIGITAL INTERACTIONS INVOLVING USER ARBITRATION BETWEEN INDIVIDUAL OUTCOME OBSERVATIONS AND ADVICE” and filed on Apr. 21, 2023, the entire contents of which is incorporated herein by reference.

### BACKGROUND

The present disclosure relates to systems and devices that facilitate and/or simulate interactions between users and their environment in the presence of social cues. In some aspects the present disclosure relates to digital therapeutic methods, systems and devices.

Therapist-assisted avatar therapy is an innovative therapy in which clinicians help voice-hearers design an audio-visual representation of the heard voice. This therapy offers face-to-face interaction with a digital representation of the voice (avatar) whose speech closely matches the pitch and tone of the persecutory voice. The clinician switches between speaking as a therapist and as an avatar and facilitates a dialogue in which the voice-hearer gradually gains control within the relationship.

This therapy has proven to be particularly effective, showing reductions in the severity of auditory verbal hallucinations (AVH) 12 weeks after treatment when compared with an active control (i.e., supportive counselling therapy). In spite of these promising results, the significant differences between the treatment and control groups were no longer evident at 24 weeks.

One of the practical limitations of avatar therapy is that it requires extensive training for clinicians to engage with the client in this digital milieu, which limits its efficacy and generalizability.

### SUMMARY

A game-like environment presented to a user displaying a set of selectable elements. The user is instructed that some selectable elements contain a reward, while others do not. The user is also provided with advice that suggests the selection of one of the selectable elements, and after selection of a particular selectable element, the presence or absence of a reward is revealed. The selectable elements each have a visual attribute selected from a set of visual attributes, enabling the user to develop a sense of which type of visual attribute tends to be rewarding or non-rewarding. The behaviour of the user is modelled using a computational behavioural model, providing model parameters that facilitate tracking of changes in the user's beliefs during gameplay. The probabilistic structure of the game may be altered based on the measured learning performance, as quantified by one or more computational model parameters.

Accordingly, in some aspects of the present disclosure, there is provided a method of facilitating digital interactions with a user in a probabilistic game-like environment that involves user arbitration between individual outcome observations and advice, the method comprising:
(i) displaying, to the user, on a display device, a plurality of selectable elements, each selectable element having an associated visual attribute selected from a plurality of visual attributes, each visual attribute having a corresponding cue probability that determines the probability of receiving a reward when a selectable element with the visual attribute is selected by the user;
(ii) evaluating the cue probability of each visual attribute to determine which selectable elements are rewarding;
(iii) evaluating an advice probability to determine whether or not advice will correctly identify a selectable element associated with a reward;
(iv) providing advice, according to the evaluated advice probability, the advice indicating, to the user, a recommended selection of a selectable element;
(v) receiving input from the user selecting one of the selectable elements;
(vi) displaying to the user whether or not the selectable element selected by the user resulted in a reward;
(vii) repeating steps (i) through (vi) one or more times, each time evaluating (a) the cue probabilities of the visual attributes according to a cue probability schedule and (b) the advice probability according to a prescribed advice probability schedule;
(viii) fitting the input received from the user to a computational model of learning and obtaining a plurality of model parameters characterizing arbitration by the user between observed reward outcomes and the advice;
(ix) employing the value of at least one parameter to modify a probabilistic structure of the game-like environment; and
(x) repeating steps (i) through (vii) while employing the modified probabilistic structure of the game-like environment, such that the probabilistic structure of the game-like environment adaptively evolves according to the user's arbitration between individual outcome observations and the advice.

In some example implementations of the method, employing the value of at least one parameter to modify a probabilistic structure of the game-like environment comprises:
determining that a parameter associated with social bias falls within a range associated with disregard of the advice; and
providing feedback to the user that is indicative of limitations in the accuracy of the advice.

Providing feedback may comprise indicating to the user that the advice is probabilistic in nature, and/or indicating an accuracy of the advice.

In some example implementations of the method, employing the value of at least one parameter to modify a probabilistic structure of the game-like environment comprises:
determining that a parameter associated with confidence of the user in determining reward locations falls within a range associated with a lack of user confidence in reward location determination; and
providing feedback to the user that is indicative of changes in the cue probability.

In some example implementations of the method, employing the value of at least one parameter to modify a probabilistic structure of the game-like environment comprises:
determining that a parameter associated with trust of the user in the advice falls within a range associated with a lack of trust of the user in the advice; and
providing feedback to the user that is indicative of limitations in accuracy of the advice.

In some example implementations of the method, employing the value of at least one parameter to modify a probabilistic structure of the game-like environment comprises:
determining that a parameter associated with volatility of cue probability falls within a range associated with excessive volatility; and
reducing a number of changes in the cue probability schedule when performing steps (i) to (vi).

In some example implementations of the method, the computational model of learning is a hierarchical Bayesian model of learning, and wherein employing the value of at least one parameter to modify a probabilistic structure of the game-like environment comprises:
determining that a parameter associated with volatility of advice probability falls within a range associated with excessive volatility; and
reducing a number of changes in the advice probability schedule when performing steps (i) to (vi).

In some example implementations of the method, employing the value of at least one parameter to modify a probabilistic structure of the game-like environment comprises employing at least one of the parameters to modify the cue probability schedule.

In some example implementations of the method, employing the value of at least one parameter to modify a probabilistic structure of the game-like environment comprises employing at least one of the parameters to modify the advice probability schedule.

In some example implementations, the method further comprises repeating steps (viii) to (x) one or more times.

In some example implementations, the method further comprises employing at least one parameter to generate and display feedback to the user.

In some example implementations of the method, the advice is provided by a digital avatar. The digital avatar may provide the advice by graphically identifying the recommended selection of the selectable element. The digital avatar may comprise one or more features selected by the user.

In some example implementations of the method, the digital avatar provides the advice in the absence of audible communication.

In some example implementations of the method, the visual attributes are colours.

In some example implementations of the method, the plurality of selectable elements comprise selectable locations in a virtual environment.

In some example implementations of the method, each visual attribute is associated with a plurality of selectable locations in the virtual environment, and wherein, when performing step (ii), the selectable locations that are rewarding are determined, for each visual attribute, by evaluation of the cue probability associated with the visual attribute.

In some example implementations of the method, the plurality of selectable elements comprise cards.

In some example implementations of the method, the plurality of selectable elements comprise doors.

In some example implementations of the method, the computational model of learning is selected from a Bayesian model of learning and a reinforcement learning model.

In another aspect, there is provided a system for facilitating digital interactions with a user in a probabilistic game-like environment that involves user arbitration between individual outcome observations and advice, the system comprising:
control circuitry comprising at least one processor and associated memory, said memory comprising instructions executable by said at least one processor for performing operations comprising:
(i) displaying, to the user, on a display device, a plurality of selectable elements, each selectable element having an associated visual attribute selected from a plurality of visual attributes, each visual attribute having a corresponding cue probability that determines the probability of receiving a reward when a selectable element with the visual attribute is selected;
(ii) evaluating the cue probability of each visual attribute to determine which selectable elements are rewarding;
(iii) evaluating an advice probability to determine whether or not advice will correctly identify a selectable element associated with a reward;
(iv) providing advice, according to the evaluated advice probability, indicating, to the user, a recommended selection of a selectable element;
(v) receiving input from the user selecting one of the selectable elements;
(vi) displaying to the user whether or not the selectable element selected by the user resulted in a reward;
(vii) repeating steps (i) through (vi) one or more times, each time evaluating (a) the cue probabilities of the visual attributes according to a cue probability schedule and (b) the advice probability according to a prescribed advice probability schedule;
(viii) fitting the input received from the user to a computational model of learning and obtaining a plurality of model parameters characterizing arbitration by the user between observed reward outcomes and the advice;
(ix) employing the value of at least one parameter to modify a probabilistic structure of the game-like environment; and
(x) repeating steps (i) through (vii) while employing the modified probabilistic structure of the game-like environment, such that the probabilistic structure of the game-like environment adaptively evolves according to the user's arbitration between individual outcome observations and the advice.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1A illustrates an example social learning game that involves user arbitration between observed outcomes and social advice based on the selection between two doors.

FIG. 1B illustrates an example cue probability schedule and an example advice probability schedule.

FIG. 1C illustrates an alternative example in which the game-like environment is rendered in the form of a virtual space (virtual world) that includes a plurality of locations which correspond to the selectable elements.

FIG. 2 illustrates an example method of facilitating digital interactions with a user in a probabilistic game-like environment that involves user arbitration between individual outcome observations and advice.

FIG. 3 illustrates an example and non-limiting computational model of learning.

FIG. 4 illustrates the tracking and display of performance metrics and model parameters.

FIG. 5 illustrates an example digital avatar that communicates the advice during the gameplay.

FIG. 6 illustrates an example computational learning and arbitration model. In this graphical notation, circles represent constants whereas hexagons and diamonds represent quantities that change in time (i.e., that carry a time/trial index). Hexagons in contrast to diamonds additionally depend on the previous state in time in a Markovian fashion. The two-branch HGF describes the generative model for advice and card probability: x₁ represents the accuracy of the current advice/card shade probability, x₂ the tendency of the advisor to offer helpful advice tendency of card shade to be rewarded, and x₃ the current volatility of the advisor's intentions/card shade probabilities. Learning parameters describe how the states evolve in time. Parameter κ determines how strongly x₂ and x₃ are coupled, and ϑ represents the meta-volatility of x₃. The response model maps the predicted shade probabilities to choices. The response model also assumes that trial-wise wagers and predictions arise from a linear combination of arbitration, informational uncertainty (advice and card), and volatility (advice and card).

FIG. 7 shows prior mean and variance of the perceptual and response model parameters for the model described in the Examples section.

FIG. 8 is a block diagram illustrating an example implementation of a computing device for performing methods disclosed herein.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

In order to overcome the shortcomings of prior attempts to implement digital therapeutic interventions for treating auditory verbal hallucinations, the present inventors developed an improved method that employs a social learning game environment to approximate client-avatar interactions by integrating advances in social learning task design and computational modelling.

Accordingly, in various example embodiments of the present disclosure, a game-like is environment presented to a user on a digital interface, such as a smartphone, tablet or computer. The user is instructed to play a reward-seeking game involving a series of turns. During each turn, the user is presented with a set of selectable elements that are graphically displayed on a user interface. The user is instructed that some of the selectable elements contain a reward, while others do not. The user is also provided with advice that suggests the selection of one of the selectable elements. After the user selects a particular selectable element, the user interface reveals to the user whether or not the selected element provides a reward.

The selectable elements each have a visual attribute selected from a set of visual attributes, such that a first set of selectable elements have a first visual attribute, a second set of selectable elements have a second visual attribute, and so forth (where there are at least two types of visual attributes). Accordingly, as the game is played, the user can develop a sense of which type of visual attribute tends to be rewarding or non-rewarding.

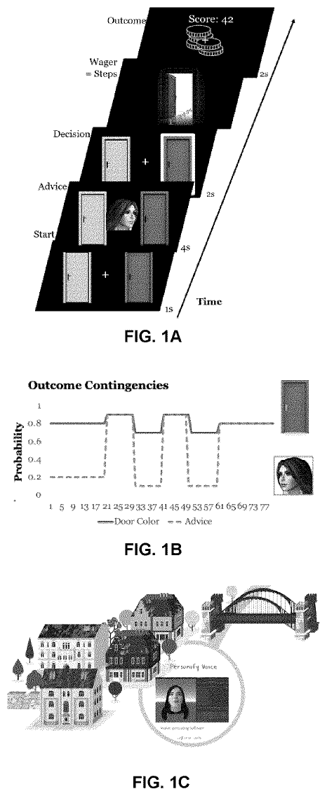

FIG. 1A illustrates one non-limiting example of a social learning game that involves user arbitration between observed outcomes and social advice based on the selection between two selectable elements that are displayed in the form of selectable doors. One of the doors is dark gray (a dark gray visual attribute) while the other door is light gray (a light gray visual attribute). During each turn, the user is presented with advice (social advice) that suggests the selection of one of the two doors. As the game is played and more turns are taken, the user learns from the social advice and determines how much to integrate the advice into the selection of a given door. The goal of the social learning game is to collect rewards (targets) that are occluded/hidden in the virtual space.

As turns are taken during the gameplay, the probability a given door shade (visual attribute) changes according to a cue probability schedule that is illustrated in FIG. 1B. The figure shows the cue probability on the vertical axis and the number of turns on the horizontal axis. When a given turn is to be taken, the system evaluates the cue probability for the turn to determine which door will be rewarding, converting the cue probability for the turn to a binary outcome such that one of the two doors is rewarding. As can be seen in the figure, after approximately 20 turns, the cue probability transitions such that the probability of the light gray door providing a reward increases from 80% to 90%.

The figure also shows how the probability of the advice being correct changes, as turns are taken, according to an advice probability schedule. The figure shows the advice probability on the vertical axis and the number of turns on the horizontal axis. When a turn is taken, the system evaluates the advice probability for the turn to determine which door will be suggested by the advice, converting the advice probability for the turn to a binary outcome such that one of the two doors is suggested. As can be seen in the figure, after approximately 20 turns, the probability of the advice being correct transitions from 20% to 90%.

The probabilistic reward learning task requires arbitration by the user between visual cues and advice. The user accrues rewards by entering (selecting) one of the two doors, which are probabilistically associated with winning points (coins). In addition to choosing the door (decision phase), the user may indicate the number of steps they are willing to take (wager phase).

The advice may be given according to a wide variety of methods. In some example implementations, the advice is provided in the form of text or graphical annotations. In some example embodiments that are described in further detail below, the advice is provided by a virtual avatar. In such example embodiments, the avatar engages with the user by indicating whether the targets are located through cues such as pointing and looking. As described below, features of the avatar may be selected by the user. In some example embodiments, the avatar provides advice in the absence of audible communication.

While FIGS. 1A and 1B illustrate an example case in which there are only two selectable elements and two corresponding visual attributes, it will be understood that both the number of selectable elements and the number of visual attributes can increase beyond two.

Furthermore, it will be understood that while the present example involves shade as the visual attribute, it will be understood that any distinguishing feature can be employed as a visual attribute. Alternative non-limiting examples of visual attributes include colour, brightness/intensity, texture, pattern, coding (e.g. text or other symbols), types (e.g. doors vs. chairs, dogs vs. cats, etc.) and animation (e.g. animated vs. non-animated or different types of animations).

While FIGS. 1A and 1B illustrate an example game-like environment involving selection between two binary selectable elements, FIG. 1C illustrates an alternative example in which the game-like environment is rendered in the form of a virtual space (virtual world) that includes a plurality of locations which correspond to the selectable elements. The locations may include selectable objects, points, shapes, or any other local feature that is selectable by the user. At least two subsets of locations (selectable elements) are rendered with different visual attributes. That is to say, a first set of objects (or features, or items) are rendered with a first visual attribute (e.g. a first colour, type, shape, pattern texture, shade, animation etc.) and a second set of objects are (or features, or items) are rendered with a second visual attribute (e.g. a first colour, type, shape, pattern texture, shade, animation etc.). In some example embodiments, three or more visual attributes may be employed.

In such an example embodiment in which multiple groups of selectable elements are spatially distributed and divided into a plurality of groups, each group being rendered with a different visual attribute, the locations that include rewards, for a given group of selectable elements, are determined by evaluating the current (i.e. for the present turn) value of the cue probability. For example, an example virtual world may include a set of ten red buildings and a set ten blue buildings, with each building colour having a respective cue probability schedule. At a given turn during the game, the cue probability for the red buildings is 50% and the cue probability for the blue buildings is 20%. It follows that five of the red buildings will be selected (e.g. randomly selected) to be rewarding and two of the blue buildings will be selected (e.g. randomly selected) to be rewarding. It is therefore apparent that example implementations of a game-like environment with groups of locations, each group having a different visual attribute, involve a spatial representation of the cue probability.

The probabilistic structure of the advice (e.g. avatar) and the rewards embedded in the environment changes in time (volatility) requiring the user to learn and update beliefs about the reliability of the advice and the association of the rewards with a given visual attribute. In some applications, the objective function may thus be to enhance or maximize interactions with the advice (e.g. avatar) and promote a collaborative (rather than competitive) exchange between the user and the advice (e.g. avatar). Accordingly, the advice preferably provides a helpful strategy overall. However, since the advice is probabilistic, it can also be wrong (i.e., randomly incorrect) about the location of the rewards. In some example implementations, with sufficient rewards (e.g. points), the user can open new worlds or environments within the app, as well as design new avatars.

During gameplay, the user arbitrates between their own mapping of the virtual space and the advice, as a function of the trust that the user places in the advice. The advice continuously provides feedback for a probabilistic identification of the rewards in the environment. As noted above, the advice does not represent full knowledge of the environment but attempts to provide helpful inputs. In the present non-limiting example, the responses are employed to measure, with the assistance of a computational model of learning (such as, but not limited to, a hierarchical Bayesian model of learning), the user's ability to detect rewards in a volatile environment, allowing the examination of the user's belief stability (e.g. trust towards the avatar, the personification of the voice they hear).

In present example method, a fixed and predefined input structure is employed, enabling real-time computational analysis of the user's behaviour in response to the advice that is provided. The behaviour of the user is modelled after each game setting (e.g. after a set of turns). By performing computational modelling in the input, the model parameters can facilitate tracking of how much the user's beliefs are changing over time. Choices and steps that were taken towards the recommended locations are modelled using a the computational model of learning (e.g. a hierarchical Bayesian model of learning) under uncertainty used to track and arbitrate between two sources of noisy information.

In some non-limiting example implementations disclosed herein, the computational model of learning is a hierarchical Bayesian model based on the hierarchical Gaussian filter, such as the model invented and introduced to computational psychiatry by Mathys and colleagues (Mathys, C., Daunizeau, J., Friston, K. J. & Stephan, K. E. A Bayesian foundation for individual learning under uncertainty. Front Hum Neurosci 5, (2011)).

However, it will be understood that the computational model of learning can take on many different forms. For example, the computational model of learning may be a Bayesian model or a reinforcement learning model, and the Bayesian model may be a hierarchical Bayesian model or a non-hierarchical Bayesian model. Non-limiting examples of suitable models include reinforcement learning models, such as the Sutton-Barto Model and the Rescorla-Wagner Model, and other example Bayesian models such as the Kalman Filter. The computational model of learning may be implemented as and/or include one or more perceptual models, response models, and mathematical formulations for arbitration.

In some example embodiments, after having completed a game, or a set of turns, the probabilistic structure of the game is altered based on the measured learning performance, as quantified by one or more computational model parameters. For example, if players display distrust or hostility towards the advice (which may be expected in some applications), cues indicating the probabilistic structure of the advice (e.g. avatar's recommendations) are cued to indicate their level of uncertainty. For example, “Avatar knows the location 70% of the time now”. Such informative cues facilitate learning about the avatar's fidelity in the game.

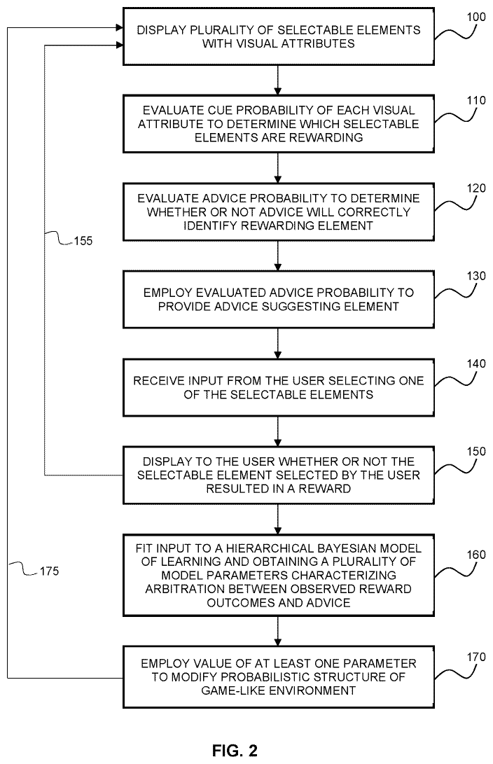

FIG. 2 illustrates an example method of facilitating digital interactions with a user in a probabilistic game-like environment that involves user arbitration between individual outcome observations and advice. In step 100, at the onset of a turn, a plurality of selectable elements are displayed to a user, each selectable element having an associated visual attribute selected from a plurality of visual attributes. Each visual attribute has a corresponding cue probability that determines the probability of receiving a reward when a selectable element with the visual attribute is selected by the user.

The cue probability of each visual attribute, as obtained from a cue probability schedule, is evaluated to determine which selectable elements are rewarding, as shown at 110. Likewise, as shown at 120, the advice probability, as obtained from an advice probability schedule, is evaluated to determine whether or not advice will correctly identify a selectable element associated with a reward. The advice, which recommends a specific selectable element, is then communicated to the user at step 130 (e.g. by the pointing or direction of gaze of a virtual avatar).

Input is then received from the user, the input selecting one of the selectable elements, as shown at 140. The user interface then displays to the user whether or not the selectable element selected by the user resulted in a reward, thus completing a turn, as shown at 150.

As shown at 155, steps 100 through 150 are repeating one or more times (i.e. one or more additional turns), each time evaluating (a) the cue probabilities of the visual attributes according to a cue probability schedule and (b) the advice probability according to a prescribed advice probability schedule.

After a set of turns have been completed (e.g. after the completion of a game consisting of several turns), the input received from the user is fitted to a computational model of learning, (such as, for example, a Bayesian model of learning or a reinforcement learning model), thereby providing a plurality of model parameters characterizing arbitration by the user between observed reward outcomes and the advice, as shown at 160. The value of at least one parameter is then employed to modify a probabilistic structure of the game-like environment, as per step 170, and step 155 is repeated one or more times, while employing the modified probabilistic structure of the game-like environment, as shown at 175. Accordingly, the probabilistic structure of the game-like environment adaptively evolves according to the user's arbitration between individual outcome observations and the advice.

### Example Computational Model

As noted above, by performing computational modelling in the input, the model parameters can facilitate tracking of how much the user's beliefs are changing over time. The computational model assumes hierarchical learning about the tendency of the advice to be helpful as well as the probability of rewards.

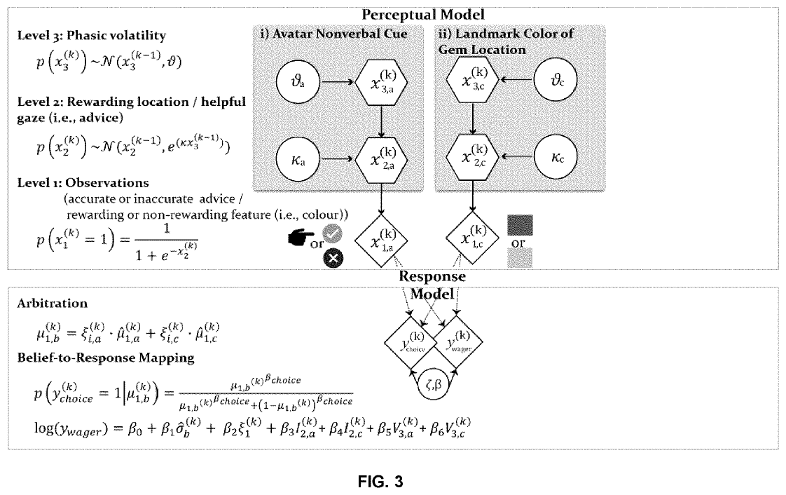

FIG. 3 illustrates an example and non-limiting computational model of learning. In this graphical notation, circles represent constants and diamonds represent quantities that change in time (i.e., that carry a time/trial index). Hexagons, like diamonds, represent quantities which change in time, but additionally depend on the previous state in time in a Markovian fashion. Two parallel HGFs describe the generative model for advice and landmark shade, where x₁ represents the accuracy of the current advice/landmark shade probability, x₂ the tendency of the adviser to offer helpful advice/tendency of the rewarding landmark shade, and x₃ the current volatility of the adviser's intentions/shade-reward probabilities. Learning parameters describe how the states evolve in time. Parameter κ determines how strongly x₂ and x₃ are coupled, and 9 represents the meta-volatility of x₃. The response model maps the predicted outcome probabilities to choices. It is assumed that trialwise steps are defined by a linear combination of arbitration, informational uncertainty (advice and shade), and volatility (advice and shade).

A detailed description of a related model that pertains to the card selection game is provided in the Examples section. It will be understood that the skilled artisan will understand how to adapt the model described in detail below to the example model structure illustrated in FIG. 3.

The fitting of a computational model of behaviour allows player-advice (avatar) interactions to adapt over time, promoting increased interactions with the advice (avatar) and providing a personalized experience for the user.

The following four modifications provide non-limiting examples that illustrate how the task can be automatically personalized based on the user's behaviour, as inferred based on the parameters of the model illustrated in FIG. 3. It will be understood that other modifications to the probabilistic structure of the game may be implemented, based on the model parameters, without departing from the intended scope of the present disclosure.

Firstly, in one example, if the parameter zeta (ζ) of the computational model is below zero (indicating that the user disregards the suggested advice), we introduce a task framing. The game is automatically adapted so that at the beginning, it reminds the user that the role of the advice is to provide helpful suggestions, but that the advice is not based on full knowledge of where the rewards are hidden, and thus may unintentionally lead to mistakes. A similar modification may be made in cases in which the parameter (κₐ) is too high, as noted below.

In another example modification, If the parameter (κ_{c}) is too high (above 1), this indicates that the user is overly uncertain about the location of the rewards, leading potentially to more errors. To reduce uncertainty, the game structure will be adapted such that the changes in the reward probabilities (locations where gems can be found) will be automatically signaled to the user using arrows. This will also show to the user that the suggestions are consistent with the actual locations of the rewards. This adaptation of the game also functions to promote trust and interaction with the advice (e.g. avatar).

In another example modification, if the parameter (κₐ) is too high (above 1), this indicates that the user is overly uncertain about the trustworthiness of the advice, leading to potential distrust. To reduce uncertainty, the game would be adapted by introducing a task framing as in the example shown above. The game is automatically adapted so that at the beginning, it reminds the user that the role of the advice is to provide helpful suggestions, but because the advice is not based on full knowledge of where the rewards are hidden, and can unintentionally involve mistakes.

In another example modification, if the meta-volatility parameters (ϑₐ, ϑ_{c},) are too high (above 0.6), the input structure of the game may be adapted to include fewer probability transitions (e.g. 4 instead of instead of 6), i.e., shifting from 20% probability, to 80%, to 20%, and then back to 80% every 20 trials. This will also ensure that the user learns the reward locations, and thus realizes that the suggestions are in general helpful, thereby promoting trust and interactivity with the advice (avatar).

The adaptation of the game, which can, in some example implementations, occur in real-time, can be used beneficially to increase social engagement with a digital avatar. This allows personalization of the game to users' behaviour and level of interaction with the avatar, and enhances user's perceived sense of control in their interactions with the avatar, promoting a sense of mastery over the game.

### Avatar

As noted above, in some example embodiments, the advice is provided by an avatar. The avatar is a sidekick in the gaming environment, as illustrated in FIG. 1C. In some example implementations, the avatar provides recommendations in the absence of audible communication. In other words, the advice provided by the avatar is communicated with nonverbal cues. This approach may be beneficial therapeutically because adding a voice component may not exactly approximate the user's internal experience of a voice; hence, reducing the ecological validity of the experience.

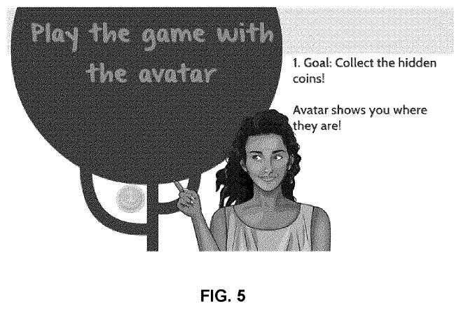

In the present non-limiting example, aspects of the present disclosure are illustrated in example implementations involving avatar therapy. By integrating social learning tasks and computational models of social interaction, the example embodiments disclosed herein facilitate the engagement with a digital avatar in an iterative social learning game as illustrated schematically in FIG. 5.

The avatar has more knowledge of the virtual environment that the user and provides advice (assistance) to the user to explore the environment in an efficient manner, allowing the user to discover and collect the rewards. In some example implementations, the help from the avatar can facilitate the uncovering of new levels and virtual environments.

### Digital Avatar Creation

In the present example, a user interface facilitates the creation of a digital avatar by a user. The avatar visually represents a personified version of a voice that the user is hearing due to an auditory verbal hallucination disorder. The creation of the avatar, and the subsequent interactions with the avatar though the game-like environment, provides the user with a sense of control over the voice, thereby providing a therapeutic benefit.

Referring again to FIG. 5, the present example method begins with an avatar creation tool, which allows the user to create the voice they hear with all features available: hair/eye colour, gender, race, face morphology and more (this step may be performed, for example, using a third-party (e.g. commercially available) software (e.g. app). Afterwards, the player is immersed in the social game environment. The avatar created joins them, and the user can decide how much or how little to cooperate with the avatar.

In some example implementations, the avatar does not speak. Instead, the avatar only engages with the user via the socially interactive game. This is because one of the challenges of avatar therapy had been to adequately match the sound of the voice experienced by the voice hearer with the voice generated by the software. The avatar thus provides recommendations in the absence of audible communication. In other words, the advice provided by the avatar is communicated with nonverbal cues. This approach may be beneficial therapeutically because adding a voice component may not exactly approximate the user's internal experience of a voice; hence, reducing the ecological validity of the experience.

### Task Performance Tracking

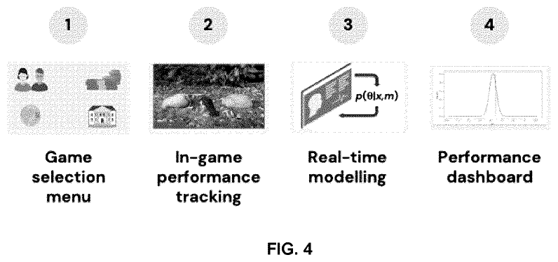

In some example implementations, performance tracking and feedback may be provided to the user. Performance metrics (accuracy, response speed, how often the player interacts with the avatar) may be are displayed, optionally along with the parameter estimates based on each game performance, as shown in FIG. 4. Users may be shown a graphical representation of their parameter estimates each time they play or complete the game.

In some example implementations, the user interface enables the user to better understand how their performance and/or parameters relate to those of other users and/or user populations. For example, users may be presented with the option of seeing their parameter estimates and where their performance lies relative to the population distribution for a given parameter (see FIG. 4). Also, in some example implementations, if the user has played the game before, the user can be presented with previous performance and parameter estimates relative to the current game. For example, by clicking on a parameter symbol, a user can receive more information about that parameter and what it reflects in terms of their performance on the game.

FIG. 4 illustrates an example implementation of real-time model parameter estimation and performance tracking. In step 1, participants choose a game from the main menu. In step 2, participants perform a first round of the game. In step 3, their choices are saved and collected for model inversion. In step 4, once participants complete a first round of the game, their performance metrics including the parameter estimates are displayed.

### Applications

The example embodiments of the present disclosure, and variations thereof, may be employed in or adapted for use with a wide variety of applications. For example, present example methods may be employed to assist mental health clinicians and clients to reduce the intensity of auditory-verbal hallucinations (e.g. for patients that suffer from psychosis and schizophrenia).

The present example embodiments, when employed by patients undergoing treatment for psychological disorders, may be beneficial in offering patients the potential to track their progress individually, without strictly relying on their therapist. In some example implementations, participants can track how performance on the game is related to reductions in symptoms and they can share their insights with their therapist.

Such applications provide a client-centered approach that can be beneficial in improving clients' wellbeing through, for example, (i) personalization (each individual can create the avatar and decide how much he/she engages with them); (ii) self-monitoring and agency: individuals can track their progress in the game and improve their score in a familiar setting; (iii) therapy, effectively providing a prescription that never runs out, in some cases permitting users to continue to engage with the digital application as long as symptoms persist.

The present example embodiments can made available to users widely, potentially providing a “prescription that does not run out” because it is not dependent on a clinician's involvement or engagement, as in the current avatar therapies.

In some example therapeutic implementations, a patient engaging in the digital application may initially play the game in the presence of (or under supervision of) their clinician/therapist, since the creation of the avatar can be in and of itself an emotional and distressing experience. Furthermore, although therapist input is not required, the application may be configured, through communication with a remote server, to facilitate sharing of their performance metrics with the therapist, to help evaluate their engagement with the avatar.

It will be understood that the example embodiments of the present disclosure may be adapted for use in a wide variety of applications in addition to those describeb above, including but not limited to, behavioural therapy for social anxiety disorders and behavioural therapy for simulating social interactions, such as, for example, teaching children perspective-taking (e.g. for use in schools and other educational settings).

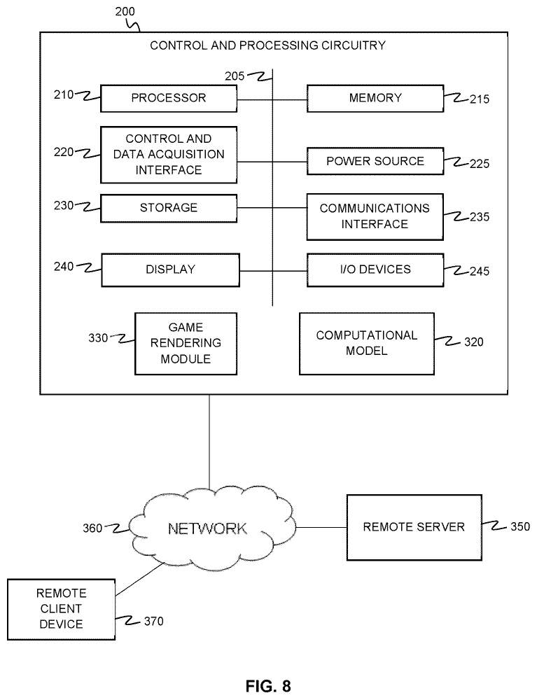

Referring now to FIG. 8, an example system is shown that includes control and processing circuitry 200 that is programmed to facilitate digital interactions with a user in a probabilistic game-like environment that involves user arbitration between individual outcome observations and advice, according to one of the example embodiments described herein, or variations thereof.

As shown in the example embodiment illustrated in FIG. 8, control and processing circuitry 200 may include a processor 210, a memory 215, a system bus 205, a data acquisition and control interface 220 for user input, a power source 225, and a plurality of optional additional devices or components such as storage device 230, communications interface 235, display 240, and one or more input/output devices 245.

The example methods described herein can be implemented, at least in part, via hardware logic in processor 210 and partially using the instructions stored in memory 215. Some example embodiments may be implemented using processor 210 without additional instructions stored in memory 215. Some embodiments may be implemented using the instructions stored in memory 215 for execution by one or more microprocessors.

For example, the example methods described herein may be implemented via processor 210 and/or memory 215. Such a methods include, and which are represented as modules, game rendering environment module 330 and computation (computational learning and arbitration model) module 320. In some example implementations, one or more aspects of the user interface (i.e. the game-like environment, the avatar creation tool, and/or performance monitoring interface features) may be generated, in whole or via the processing and control circuitry 200, or, for example, via a remote computing system that is connectable to the processing and control circuitry 200.

As shown in the example embodiment illustrated in FIG. 8, a remote client device 370 (e.g. a smartphone or computer) may be employed to permit an external user (e.g. a healthcare provider) to access data associated with the user's performance in the game, for example, to monitor the performance and/or progress of a subject in an unsupervised environment.

It is to be understood that the example system shown in the figure is not intended to be limited to the components that may be employed in a given implementation. For example, the processing and computing circuitry 200 may include a mobile computing device, such as a tablet or smartphone. In another example implementation, a portion of the control and processing circuitry 200 may be implemented, at least in part, on a remote computing system that connects to a local processing hardware via a remote network, such that some aspects of the processing are performed remotely (e.g. in the cloud), as noted above.

Although only one of each component is illustrated in FIG. 8, any number of each component can be included. For example, a computer typically contains a number of different data storage media. Furthermore, although the bus 210 is depicted as a single connection between all of the components, it will be appreciated that the bus 210 may represent one or more circuits, devices or communication channels which link two or more of the components. For example, in many computers, bus 210 often includes or is a motherboard.

Although some example embodiments of the present disclosure can be implemented in fully functioning computers and computer systems, various embodiments are capable of being distributed as a computing product in a variety of forms and are capable of being applied regardless of the particular type of machine or computer readable media used to actually effect the distribution.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

### Examples

The following examples are presented to enable those skilled in the art to understand and to practice embodiments of the present disclosure. They should not be considered as a limitation on the scope of the disclosure, but merely as being illustrative and representative thereof.

### Example Learning Model: Hierarchical Gaussian Filter

The Hierarchical Gaussian Filter (HGF) is an example model of hierarchical Bayesian inference widely used for computational analyses of behaviour (e.g., (Iglesias et al., 2013; Vossel et al., 2013; Hauser et al., 2014; de Berker et al., 2016; Marshall et al., 2016). To apply it to the task of card/door selection illustrated in FIG. 1A, it is assumed that the rewarded card shade (individual learning) and the advice accuracy (social learning) varied as a function of hierarchically coupled hidden states:

$${x}_{1}^{( k )} , {x}_{2}^{( k )} , … , {x}_{n}^{( k )} .$$

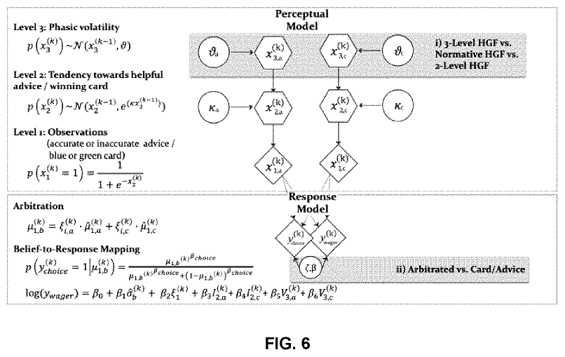

They evolved in time by performing Gaussian random walks. At every level, the step size was controlled by the state of the next-higher level (FIG. 6).

Starting from the bottom of the hierarchy, states x_{1,a} and x_{1,c} represented binary variables, namely the advice accuracy (1 for accurate, 0 for inaccurate) and the rewarded card shade (1 for light gray, 0 for green). All states higher than x₁ were continuous. They denoted (i) the advisor fidelity and tendency for a given card shade to be rewarded, and (ii) the rate of change of the advisor's intentions and card shade contingencies, respectively. Four learning parameters, namely, κₐ, κ_{c}, ϑₐ and ϑ_{c} determined how quickly the hidden states evolved in time. Parameter κ represented the degree of coupling between the second and the third levels in the hierarchy, whereas ϑ determined the variability of the volatility over time (meta-volatility). This constitutes the generative model of the process producing the outcomes observed by participants. The overall model and the formal equations describing these relations in a social learning context are detailed in Diaconescu et al., 2020 (Andreea Oliviana DiaconescuMadeline StecyLars KasperChristopher J BurkeZoltan NagyChristoph MathysPhilippe N Tobler (2020) Neural arbitration between social and individual learning systems eLife 9:e54051; https://doi.org/10.7554/eLife.54051).

Model Inversion: Agent-Specific arbitration

In accordance with Bayes' rule, we assumed that participants who make inferences on advice and card shades form posterior beliefs over the hidden states (i.e., congruency of advice with actual card shade; rewarded card shade) based on the outcomes they observe. Model inversion is the application of Bayes' rule to a generative model such as the one described above. This leads to a perceptual model, which describes participants' beliefs about hidden states. Assuming Gaussian distributions, these agent-specific beliefs are denoted by their summary statistics, i.e., μ (mean) and σ (variance/uncertainty) or the inverse of the variance π=1/σ (precision/certainty).

Using variational Bayes under the mean-field approximation, simple analytical trial-by-trial update equations can be derived. The posterior means

$${μ}_{i}^{( k )}$$

or predictions on each trial k at each level of the hierarchy i change as a function of precision-weighted prediction errors (PEs): The

$$Δ {μ}_{i}^{( k )} ∝ \frac{{\hat{π}}_{i - 1}^{( k )}}{{π}_{i}^{( k )}} {δ}_{i - 1}^{( k )} \qquad ( 1 )$$

Throughout, predictions or prior beliefs about the hidden states (before observing the outcome) are denoted with a hat symbol. States

$${\hat{π}}_{i - 1}^{( k )} and {π}_{i}^{( k )}$$

represent the estimated precisions about (i) the input from the level below (i.e., precision of the data-advice congruency or rewarded card shade) and (ii) the belief at the current level, respectively.

The updates about the advisor's fidelity are:

$$Δ {μ}_{2 , a}^{( k )} = \frac{1}{{π}_{2 , a}^{( k )}} {δ}_{1 , a}^{( k )} \qquad ( 2 )$$

$$where$$

$${δ}_{1 , a}^{( k )} = {u}^{( k )} - {\hat{μ}}_{1 , a}^{( k )} . \qquad ( 3 )$$

Variable u^{(k)} is the sensory input at trial k, where given advice is either accurate (u^{(k)}=1) or inaccurate (u^{(k)}=0). Furthermore,

$${\hat{μ}}_{1 , a}^{( k )}$$

corresponds to the logistic sigmoid of the current expectation of the advisor fidelity:

$${\hat{μ}}_{1 , a}^{( k )} = s ( {μ}_{2 , a}^{( k - 1 )} ) = \frac{1}{1 + exp ( - {μ}_{2 , a}^{( k - 1 )} )} . \qquad ( 4 )$$

The current belief precision is equivalent to:

$${π}_{2 , a}^{( k )} = {\hat{π}}_{2 , a}^{( k )} + \frac{1}{{\hat{π}}_{1 , a}^{( k )}} \qquad ( 5 )$$

with the predicted (i) belief precision

$${\hat{π}}_{2 , a}^{( k )}$$

and (ii) the sensory, lower-level precision about the advice

$${\hat{π}}_{1 , a}^{( k )}$$

computed as:

$${\hat{π}}_{2 , a}^{( k )} = \frac{1}{\frac{1}{{π}_{2 , a}^{( k - 1 )}} + exp ( κ {μ}_{3 , a}^{( k - 1 )} + ω )} \qquad ( 6 )$$

$${\hat{π}}_{1 , a}^{( k )} = \frac{1}{{\hat{μ}}_{1 , a}^{( k )} ( 1 - {\hat{μ}}_{1 , a}^{( k )} )} . \qquad ( 7 )$$

Thus, the advice belief precision depends on (i) the predicted sensory precision of the input,

$${\hat{π}}_{1}^{( k )}$$

and (ii) the predicted volatility,

$${μ}_{3 , a}^{( k - 1 )}$$

from the level above via equation 6.

The precision-weighted PE about the advice, which is used to update the belief about fidelity is equivalent to:

$${ε}_{2 , a} = \frac{1}{{π}_{2 , a}^{( k )}} {δ}_{1 , a}^{( k )} \qquad ( 8 )$$

Going up the hierarchy, the updates of advice volatility are proportional to precision-weighted PEs:

$$Δ {μ}_{3}^{( k )} ∝ \frac{1}{{π}_{3}^{( k )}} {δ}_{2 , a}^{( k )} . \qquad ( 9 )$$

They depend on the higher-level volatility PE δ_{2,a}:

$${δ}_{2 , a}^{( k )} = \frac{{\hat{π}}_{2 , a}^{( k )}}{{π}_{2 , a}^{( k )}} + {( {π}_{2 , a}^{( k )} )}^{2} {{\hat{π}}_{2 , a}^{( k )} ( Δ {μ}_{2 , a}^{( k )} )}^{2} - 1 , \qquad ( 10 )$$

and the higher-level volatility precision π₃:

$${π}_{3 , a}^{( k )} = {\hat{π}}_{3 , a}^{( k )} + \frac{1}{2} {( {γ}_{2 , a}^{( k )} )}^{2} + {( {γ}_{2 , a}^{( k )} )}^{2} {δ}_{2 , a}^{( k )} - \frac{1}{2} {γ}_{2 , a}^{( k )} {δ}_{2 , a}^{( k )} , \qquad ( 11 )$$

with the precision of the prediction about volatility given by

$${\hat{π}}_{3 , a}^{( k )} = \frac{1}{\frac{1}{{π}_{3 , a}^{( k - 1 )}} + {ϑ}_{a}} . \qquad ( 12 )$$

The third level, the precision-weighted volatility PE is equivalent to:

$${ε}_{3 , a} = \frac{1}{{π}_{3}^{( k )}} {δ}_{2 , a}^{( k )} . \qquad ( 13 )$$

The same form of update equations (and precision-weighted PEs) can be derived for the individual information source, updating beliefs about the rewarded card shade, i.e.:

$${ε}_{2 , c} = \frac{1}{{π}_{2 , c}^{( k )}} {δ}_{1 , c}^{( k )} \qquad ( 14 )$$

$$and$$

$${ε}_{3 , c} = \frac{1}{{π}_{3 , c}^{( k )}} {δ}_{2 , c}^{( k )} . \qquad ( 15 )$$

The prediction errors exhibit a similar form as for the advice, with

$${δ}_{1 , c}^{( k )} = {u}^{( k )} - {\hat{μ}}_{1 , c}^{( k )} \qquad ( 16 )$$

for the outcome PE and

$${δ}_{2 , c}^{( k )} = \frac{{\hat{π}}_{2 , c}^{( k )}}{{π}_{2 , c}^{( k )}} + {( {π}_{2 , c}^{( k )} )}^{2} {{\hat{π}}_{2 , c}^{( k )} ( Δ {μ}_{2 , c}^{( k )} )}^{2} - 1 \qquad ( 17 )$$

for the card volatility PE. The individually estimated card shade probability is equivalent to the logistic sigmoid of the current expectation of the rewarding card shade:

$${\hat{μ}}_{1 , c}^{( k )} = s ( {μ}_{2 , c}^{( k - 1 )} ) = \frac{1}{1 + exp ( - {μ}_{2 , c}^{( k - 1 )} )} . \qquad ( 18 )$$

In this context, Bayes-optimality is individualized with respect to the values of the learning parameters, which were allowed to differ across participants.

### Arbitration Signal

As described in Diaconescu et al., (2020), within this computational framework, arbitration is defined as the relative perceived precision associated with each information source, which is equivalent to the precision of the prediction of each information channel (advice or card; i.e., \hat{π}) divided by the total precision. Arbitration is consistent with Bayes' rule representing the optimal integration of the two inferred states by their precisions.

Arbitration towards advice—i.e., the perceived reliability of the social information source is equivalent to:

$${ξ}_{i , a}^{( k )} = \frac{ζ {\hat{π}}_{i , a}^{( k )}}{ζ {\hat{π}}_{i , a}^{( k )} + {\hat{π}}_{i , c}^{( k )}} \qquad ( 19 )$$

on each trial k at each level of the hierarchy i with ζ as the social bias or the additional bias towards the advice.

At the first level and at i=1, the participant relies preferentially on the social input during action selection when

$${ξ}_{1 , a}^{( k )}$$

exceeds 0.5. Conversely, when

$${ξ}_{1 , a}^{( k )}$$

is below 0.5 (see Eq. 9), the participant relies more on individual (estimates of) card shade probabilities:

$${ξ}_{1 , c}^{( k )} = \frac{{\hat{π}}_{1 , c}^{( k )}}{ζ {\hat{π}}_{1 , a}^{( k )} + {\hat{π}}_{1 , c}^{( k )}} = 1 - {ξ}_{1 , a}^{( k )} \qquad ( 20 )$$

### Response Model

To map beliefs to decisions, it was assumed that the prediction of card shade on a given trial k is a function of arbitration and of the predictions afforded by each source (see Eq. 21). The response model predicts two components of the behavioural response: (i) the participant's decision to accept or reject the advice and (ii) the number of points wagered on every trial. Responses were coded as y=1 when participants took the advice and chose the card shade indicated by the advisor, and y=0 when participants decided against following the advice and chose the opposite card shade. The expected outcome probability is thus a precision-weighted sum of the two information sources, the estimates of advice accuracy and rewarding shade probability.

$${μ}_{1 , b}^{( k )} = {ξ}_{i , a}^{( k )} \cdot {\hat{μ}}_{1 , a}^{( k )} + {ξ}_{1 , c}^{( k )} \cdot {\hat{μ}}_{1 , c}^{( k )} \qquad ( 21 )$$

where

$${ξ}_{i , a}^{( k )} and {ξ}_{1 , c}^{( k )}$$

are the arbitration for each information source;

$${\hat{μ}}_{1 , a}^{( k )}$$

is the expected advice accuracy (Eq. 4) and

$${\hat{μ}}_{1 , c}^{( k )}$$

is the transformed expected card shade probability from the perspective of the advice (i.e., the estimated card shade probability indicated by the advisor).

It follows from Eqn 21, that social weighting is represented by the first term of this integrated sum—i.e,

$${ξ}_{i , a}^{( k )} \cdot {\hat{μ}}_{1 , a}^{( k )}$$

whereas card shade weighting is represented by the second term or

$${ξ}_{1 , c}^{( k )} \cdot {\hat{μ}}_{1 , c}^{( k )} .$$

The probability that participants chose a particular card shade according to their expectations about the outcome (Equation 21) was modelled by a softmax function:

$$p ( {y}_{choice}^{( k )} = 1 \vert {\hat{μ}}_{1 , b}^{( k )} ) = \frac{{\hat{μ}}_{1 , b}^{{( k )}^{{β}_{choice}}}}{{\hat{μ}}_{1 , b}^{{( k )}^{{β}_{choice}}} + {( 1 - {\hat{μ}}_{1 , b}^{( k )} )}^{{β}_{choice}}} \qquad ( 22 )$$

where β_{choice}>0 is the participant-specific inverse decision temperature parameter. A low decision temperature (high β_{choice}) means always choosing the highest probability shade, whereas a high decision temperature (low β_{choice}) means sampling randomly from a uniform distribution.

The number of points wagered provided us with a behavioural readout of decision confidence. It was intended to formally explain trial-wise wager responses as a linear function of various sources of uncertainty and precision associated with the lottery outcome prediction: (i) irreducible decision uncertainty or ap (k) about the outcome, (ii) arbitration, (iii) informational uncertainty about the card shade or the advice, and (iv) environmental uncertainty/volatility about the card shade or the advice. We transformed these computational quantities down to the first level in the hierarchy using the sigmoid transformation and used them to predict the trial-by-trial wager:

$$log ( {y}_{wager} ) = {β}_{0} + {β}_{1} {\hat{σ}}_{b}^{( k )} + {β}_{2} {ξ}_{1}^{( k )} + {β}_{3} {I}_{2 , a}^{( k )} + {β}_{4} {I}_{2 , c}^{( k )} + {β}_{5} {V}_{3 , a}^{( k )} + {β}_{6} {V}_{3 , c}^{( k )} \qquad ( 23 )$$

$$with$$

$${\hat{σ}}_{b}^{( k )} = {\hat{μ}}_{1 , b}^{( k )} ( 1 - {\hat{μ}}_{1 , b}^{( k )} ) . \qquad ( 24 )$$

Parameter ζ captures the social bias in arbitration (equation 19) and

$${I}_{2 , a}^{( k )}$$

is the informational uncertainty about the advisor fidelity:

$${I}_{2 , a}^{( k )} = {\hat{μ}}_{1 , a}^{( k )} ( 1 - {\hat{μ}}_{1 , a}^{( k )} ) {\hat{σ}}_{2 , a}^{( k )} \qquad ( 25 )$$

where

$${\hat{σ}}_{2 , a}^{( k )}$$

is the inverse of

$${\hat{π}}_{2 , a}^{( k )}$$

and represents the information uncertainty of the prediction about the advisor's fidelity (Eq. 6).

The environmental volatility is defined as:

$${V}_{3 , a}^{( k )} = {\hat{μ}}_{1 , a}^{( k )} ( 1 - {\hat{μ}}_{1 , a}^{( k )} ) exp ( {μ}_{3 , a}^{( k - 1 )} ) . \qquad ( 26 )$$

Equivalent equations can be derived for the individual information source.

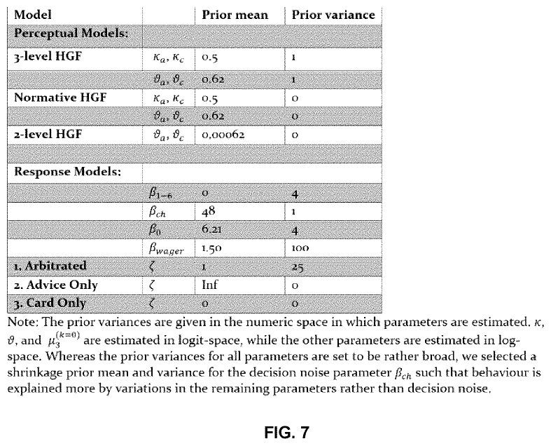

The trial-wise wager amount predicted by the model is then defined as:

$${\hat{y}}_{wager} {=}^{def} log ( {y}_{wager} ) + \sqrt{{β}_{wager}} \qquad ( 27 )$$

where β_{wager} is a stochasticity parameter associated with the wager amount. For the priors of all β parameters estimated here, refer to FIG. 7.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of facilitating digital interactions with a user in a probabilistic game-like environment that involves user arbitration between individual outcome observations and advice, the method comprising:
(i) displaying, to the user, on a display device, a plurality of selectable elements, each selectable element having an associated visual attribute selected from a plurality of visual attributes, each visual attribute having a corresponding cue probability that determines the probability of receiving a reward when a selectable element with the visual attribute is selected by the user;
(ii) evaluating the cue probability of each visual attribute to determine which selectable elements are rewarding;
(iii) evaluating an advice probability to determine whether or not advice will correctly identify a selectable element associated with a reward;
(iv) providing advice, according to the evaluated advice probability, the advice indicating, to the user, a recommended selection of a selectable element;
(v) receiving input from the user selecting one of the selectable elements;
(vi) displaying to the user whether or not the selectable element selected by the user resulted in a reward;
(vii) repeating steps (i) through (vi) one or more times, each time evaluating (a) the cue probabilities of the visual attributes according to a cue probability schedule and (b) the advice probability according to a prescribed advice probability schedule;
(viii) fitting the input received from the user to a computational model of learning and obtaining a plurality of model parameters characterizing arbitration by the user between observed reward outcomes and the advice;
(ix) employing the value of at least one parameter to modify a probabilistic structure of the game-like environment; and
(x) repeating steps (i) through (vii) while employing the modified probabilistic structure of the game-like environment, such that the probabilistic structure of the game-like environment adaptively evolves according to the user's arbitration between individual outcome observations and the advice.

2. The method according to claim 1 wherein employing the value of at least one parameter to modify a probabilistic structure of the game-like environment comprises:
determining that a parameter associated with social bias falls within a range associated with disregard of the advice; and
providing feedback to the user that is indicative of limitations in the accuracy of the advice.

3. The method according to claim 2 wherein providing feedback comprises indicating to the user that the advice is probabilistic in nature.

4. The method according to claim 2 wherein providing feedback comprises indicating an accuracy of the advice.

5. The method according to claim 1 wherein employing the value of at least one parameter to modify a probabilistic structure of the game-like environment comprises:
determining that a parameter associated with confidence of the user in determining reward locations falls within a range associated with a lack of user confidence in reward location determination; and
providing feedback to the user that is indicative of changes in the cue probability.

6. The method according to claim 1 wherein employing the value of at least one parameter to modify a probabilistic structure of the game-like environment comprises:
determining that a parameter associated with trust of the user in the advice falls within a range associated with a lack of trust of the user in the advice; and
providing feedback to the user that is indicative of limitations in accuracy of the advice.

7. The method according to claim 1 wherein employing the value of at least one parameter to modify a probabilistic structure of the game-like environment comprises:
determining that a parameter associated with volatility of cue probability falls within a range associated with excessive volatility; and
reducing a number of changes in the cue probability schedule when performing steps (i) to (vi).

8. The method according to claim 1 wherein the computational model of learning is a hierarchical Bayesian model of learning, and wherein employing the value of at least one parameter to modify a probabilistic structure of the game-like environment comprises:
determining that a parameter associated with volatility of advice probability falls within a range associated with excessive volatility; and
reducing a number of changes in the advice probability schedule when performing steps (i) to (vi).

9. The method according to claim 1 wherein employing the value of at least one parameter to modify a probabilistic structure of the game-like environment comprises employing at least one of the parameters to modify the cue probability schedule.

10. The method according to claim 1 wherein employing the value of at least one parameter to modify a probabilistic structure of the game-like environment comprises employing at least one of the parameters to modify the advice probability schedule.

11. The method according to claim 1 further comprising repeating steps (viii) to (x) one or more times.

12. The method according to claim 1 further comprising employing at least one parameter to generate and display feedback to the user.

13. The method according to claim 1 wherein the advice is provided by a digital avatar.

14. The method according to claim 13 wherein the digital avatar provides the advice by graphically identifying the recommended selection of the selectable element.

15. The method according to claim 13 wherein the digital avatar comprises one or more features selected by the user.

16. The method according to claim 13 wherein the digital avatar provides the advice in the absence of audible communication.

17. The method according to claim 1 wherein the visual attributes are colours.

18. The method according to claim 1 wherein the plurality of selectable elements comprise selectable locations in a virtual environment.

19. The method according to claim 18 wherein each visual attribute is associated with a plurality of selectable locations in the virtual environment, and wherein, when performing step (ii), the selectable locations that are rewarding are determined, for each visual attribute, by evaluation of the cue probability associated with the visual attribute.

20. The method according to claim 1 wherein the plurality of selectable elements comprise cards.

21. The method according to claim 1 wherein the plurality of selectable elements comprise doors.

22. The method according to claim 1 wherein the computational model of learning is selected from a Bayesian model of learning and a reinforcement learning model.

23. A system for facilitating digital interactions with a user in a probabilistic game-like environment that involves user arbitration between individual outcome observations and advice, the system comprising:
control circuitry comprising at least one processor and associated memory, said memory comprising instructions executable by said at least one processor for performing operations comprising:
(i) displaying, to the user, on a display device, a plurality of selectable elements, each selectable element having an associated visual attribute selected from a plurality of visual attributes, each visual attribute having a corresponding cue probability that determines the probability of receiving a reward when a selectable element with the visual attribute is selected;
(ii) evaluating the cue probability of each visual attribute to determine which selectable elements are rewarding;
(iii) evaluating an advice probability to determine whether or not advice will correctly identify a selectable element associated with a reward;
(iv) providing advice, according to the evaluated advice probability, indicating, to the user, a recommended selection of a selectable element;
(v) receiving input from the user selecting one of the selectable elements;
(vi) displaying to the user whether or not the selectable element selected by the user resulted in a reward;
(vii) repeating steps (i) through (vi) one or more times, each time evaluating (a) the cue probabilities of the visual attributes according to a cue probability schedule and (b) the advice probability according to a prescribed advice probability schedule;
(viii) fitting the input received from the user to a computational model of learning and obtaining a plurality of model parameters characterizing arbitration by the user between observed reward outcomes and the advice;
(ix) employing the value of at least one parameter to modify a probabilistic structure of the game-like environment; and
(x) repeating steps (i) through (vii) while employing the modified probabilistic structure of the game-like environment, such that the probabilistic structure of the game-like environment adaptively evolves according to the user's arbitration between individual outcome observations and the advice.
